(19) United States
(12) Patent Application Publication
(10) Pub. No.: US 2022/0378638 A1
(43) Pub. Date: Dec. 1, 2022

(54) AEROMEDICAL BIO-CONTAINMENT MODULE

(71) Applicant: KNIGHT AEROSPACE, San Antonio, TX (US)

(72) Inventors: Michael KNIGHT, San Antonio, TX (US); Bianca RHODES, San Antonio, TX (US)

(73) Assignee: KNIGHT AEROSPACE, San Antonio, TX (US)

(21) Appl. No.: 17/824,084

(22) Filed: May 25, 2022

Related U.S. Application Data
(60) Provisional application No. 63/194,782, filed on May 28, 2021.

(51) Int. Cl. A61G 10/02; B65D 88/14; B65D 90/02; B65D 88/74; B01D 46/00; A61M 16/10

(52) U.S. Cl. CPC A61G 10/023; B65D 88/14; B65D 90/026; B65D 88/74; B01D 46/0028; A61M 16/1005; B01D 2273/30; B01D 2279/50; B01D 2279/65

(57) ABSTRACT

Embodiments provide an aeromedical bio-containment module for use in a cargo plane. The aeromedical bio-containment module includes a fully integrated pallet system allowing for the module to roll onto the cargo plane. An integrated set of lugs allows the module to be locked into the cargo plane floor without the use of chains or other pallets. The aeromedical bio-containment module features a ward area for safe treatment of patients, an anteroom, and an office area for personnel. The aeromedical bio-containment module operates under a negative pressure system, and includes a full air filtration system to remove pathogens, particulates, and other airborne contaminants.

### CROSS REFERENCE TO RELATED APPLICATIONS

This application is related to, and claims the priority to, U.S. Provisional Patent Application Ser. No. 63/194,782, entitled, “AEROMEDICAL BIO-CONTAINMENT MODULE,” filed on May 28, 2021, which is hereby incorporated by reference in its entirety into this application.

### BACKGROUND

Embodiments relate to a medical bio-containment module. More specifically, embodiments relate to a modular, bio-isolated system with advanced negative pressure and filtration system for use on cargo aircraft.

### DESCRIPTION OF RELATED ART

Air transport of medically fragile, injured, sick, or infectious patients is important during and after natural disasters, times war, pandemics, epidemics, and the like. Long-distance transport of multiple patients has generally been done through military or civilian cargo planes. Maintaining isolation of the patient area, including isolation of air flow, is critical when patients carry potentially infectious diseases, especially diseases caused by airborne pathogens. Additionally, systems used to transport medical patients in cargo planes must be attached to the cargo plane beds. Conventional modular systems use pallets to hold shipping containers acting as rooms, or other structures, such as transport rooms. These pallets and containers or transport rooms are held to the pallet and the cargo plane via chains or straps. These conventional systems require additional systems to secure the structure to the plane, and do not provide appropriate amenities to support patients on long-haul flights. Therefore, a system that is fully integrated, biologically contained, providing full medical support technology, and is easy to load and secure to the cargo plane is needed.

### SUMMARY

The various embodiments disclosed herein provide a fully contained module capable of being rolled onto a cargo plane, locked into place, and hooked into electrical systems to provide a safe and secure patient transport system for a plane. The module operates under a negative air pressure, and contains a filtration system capable of removing particulates, pathogens, and other air containments from inside the module to ensure that air containments do not exit the system. The embodiments disclosed herein allow for safe transport of medical personnel and patients without endangering other passengers or plane crew. The medical bio-containment module has multiple areas, segregated within the system, and capable of isolation. The ward area is kept under negative pressure with all contaminated air exiting the area being filtered through a filtration system. The anteroom connected to the ward area allows for personnel to exit and enter the ward area. The anteroom is also under the negative pressure air filtration system, but once sealed and all air has been removed and replaced with clean air in the anteroom, the anteroom can open into the office area. The office area allows personnel to work while monitoring conditions in the ward area. Bathrooms, laboratory areas, storage, and cleaning areas are also provided within the module.

An object of the various embodiments is to provide a self-contained module that allows for patients and personnel to be safely transported without endangering the plane crew or other passengers. The anteroom in the module also protects the personnel from infection as the system allows for decontamination and removal of personal protective equipment (PPE) while protecting the personnel by keeping segregation and filtering all air through a filtration system. Another object of the various embodiments is to be an air-safe, fully contained module allowing for safe and easy transport of medical patients and personnel, with a fully integrated system which allows the module to be easily pulled or pushed onto cargo planes, a pallet system capable of interacting with the locking system so that the module is locked into place safely onto the cargo plane without chains or straps, and an electrical system to connect to the cargo plane's electrical system. The module is entirely self-contained, and no modifications or attachments are required to integrate the module into the cargo planes.

Disclosed herein is a medical bio-containment module for use in a cargo plane. The medical bio-containment module includes a pallet system, which includes a flat pallet bottom, operable to allow the medical bio-containment module to be rolled on and rolled off of the cargo plane utilizing existing rollers on a floor of the cargo plane; four walls, a floor, and a ceiling, constructed so that an airtight module interior is formed and an outer structure of the medical bio-containment module is generated; a negative pressure fan system, operable to maintain a negative pressure within the airtight module interior and operable to, when engaged, remove contaminated air from the medical bio-containment module; and an air filtration system connected to the negative pressure fan system, operable to remove airborne pathogens and particulates from the air.

The medical bio-containment module also includes an anteroom, which includes an office door and an anteroom door, where the anteroom is contained under negative pressure from the negative pressure fan system; a ward area, which includes the anteroom door and a patient bed, a first chair, and medical equipment, also contained under negative pressure from the negative pressure fan system; and an office area, which includes the office door and an access door. In some embodiments, the office area is not contained under negative pressure from the negative pressure fan system. In some embodiments, the office area is contained under negative pressure from the negative pressure fan system.

In some embodiments, the medical bio-containment module is operable to maintain negative air pressure in designated sections of the airtight module interior such as the ward area and the anteroom through the negative pressure fan system while the cargo aircraft is in flight and while the cargo aircraft is on the ground. In some embodiments, the outer structure of the medical bio-containment module is made of aircraft grade aluminum. In some embodiments, the airtight module interior includes acoustic reduction materials operable to reduce noise levels. In some embodiments, the pallet system also includes a plurality of lugs, operable to allow a locking mechanism of the cargo plane to interact with the lugs and lock the medical bio-containment module in place on the cargo plane. In further embodiments, no chains are used to lock the medical bio-containment module in place on the cargo plane.

In some embodiments, the medical bio-containment module includes a decompression panel operable to blow out from the outer structure during a decompression emergency allowing air flow through the airtight module interior. In some embodiments, the medical bio-containment module includes an access panel operable to, when opened, allow access to a main landing gear in an C-130J cargo plane.

In some embodiments, the medical bio-containment module meets or exceeds FAA FAR 25 regulations. In some embodiments, the chair and the patient bed are FAA approved systems. In some embodiments, the medical bio-containment module includes an electrical system operable to provide power to the medical bio-containment module, and further where the electrical system uses MIL-STD or DO-160 approved equipment.

In some embodiments, the air filtration system includes dust filters and HEPA filters. In some embodiments, the medical bio-containment module includes a backup power system operable to power the medical bio-containment module in the event of a cargo plane power source is lost. In some embodiments, the medical equipment includes a liquid oxygen system operable to provide up to 6 patients with enough oxygen for a 15 hour mission.

Further disclosed is a method of using the medical bio-containment module including the steps of rolling the medical bio-containment module onto the cargo aircraft; locking the medical bio-containment module onto the cargo plane by engaging the locking system on the cargo plane such that the locking system interacts with a plurality of lugs, the pallet system including the plurality of lugs; connecting the electrical system from the medical bio-containment module to a cargo aircraft electrical system; loading patients and staff into the ward area; shutting the access door, the office door and the anteroom door; and engaging the negative pressure fan system. In some embodiments, the step of engaging the locking system is performed in the absence of the use of chains or straps.

### BRIEF DESCRIPTION OF DRAWINGS

FIG. 1 is a representation of the exterior of the medical bio-containment module, according to an embodiment.

FIG. 2 is a representation of the interior layout of the medical bio-containment module, according to an embodiment.

FIG. 3 is a representation of the pallet system, according to an embodiment.

FIG. 4 is a representation of a side view of an edge of the pallet system, according to an embodiment.

FIG. 5 is a photograph of the medical bio-containment module loaded into a cargo aircraft, according to an embodiment.

FIG. 6 is a photograph of the medical bio-containment module being loaded into a cargo aircraft, according to an embodiment.

FIG. 7 is an alternate view of a representation of the pallet system, according to an embodiment.

### DETAILED DESCRIPTION

Modes for carrying out the various embodiments will now be described, but the invention is not intended to be limited to the following embodiments. It should be understood that those in which changes, improvements, or the like are appropriately added to the following embodiments based on ordinary knowledge of a person skilled in the art are also included in the scope of the various embodiments without departing from the spirit of the invention.

The aeromedical bio-containment module is a medical bio-containment module for use in cargo planes that is fully contained and capable of being isolated from the rest of the cargo plane, with a negative air pressure system and a filtration system that removes particulates, pathogens, and other air containments from inside the module to ensure that air containments do not exit the module. The medical bio-containment module can be used for patients afflicted with infectious diseases, critical patient care, ambulatory patient care, and passenger transportation. The medical bio-containment module can be utilized in multiple models of cargo aircraft, including the C-130, C-130J, C-17, C-390, A-400M, and C5 aircraft. The various embodiments disclosed herein provide a fully contained module capable of being rolled onto a cargo plane, locked into place, and hooked into electrical systems to provide a safe and secure system for a plane. The medical bio-containment module has an exterior and an interior area. The medical bio-containment module has multiple interior areas, segregated within the system, and capable of isolation. The ward area is kept under negative pressure with all contaminated air exiting the area being filtered through a filtration system. The anteroom connected to the ward area allows for personnel to exit and enter the ward area. The anteroom is also under the negative pressure air filtration system, but once sealed and all air has been removed and replaced with clean air in the anteroom, the anteroom can open into the office area. The office area allows personnel to work while monitoring conditions in the ward area. Bathrooms, laboratory areas, storage, and cleaning areas are also provided within the module.

The medical bio-containment module is comprised of (A) the structure, (B) the ward area, (C) the anteroom, and (D) the office area.

### (A) Structure

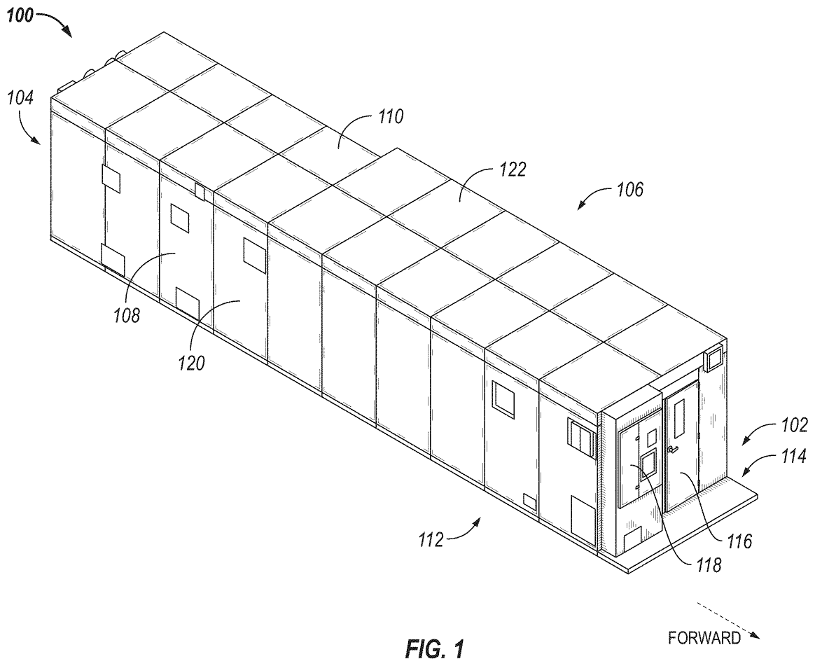

The exterior system provides the structure of the medical bio-containment module. An embodiment of the medical bio-containment module 100 is shown in FIG. 1. The medical bio-containment module 100 includes the front end 102, the back end 104, and first side 106, and the second side 108. The front end 102, the back end 104, and first side 106, and the second side 108 all are connected and sealed to the top side 110 and the bottom side 112, forming a rectangular prism structure, fully sealed and contained. This generates an airtight module interior of the medical bio-containment module and an exterior, or an outer structure, of the medical bio-containment module. The structural support systems for the medical bio-containment module are made of aircraft-grade aluminum, as are the sides, top, bottom, and doors. In some embodiments, the structure is mechanically fastened together with military aircraft grade hardware, and not welded. Beneficially, the lack of welding improves manufacturing speed and cost. In some embodiments, the module is 39.5 feet long from front end 102 to back end 104. All materials and hardware is aircraft grade. The front end 102 includes the access doors 116 and the electrical cabinet 118. The access doors 116 open into the office area (not shown) in the interior of the medical bio-containment module, and allow for personnel, crew, or patient access from the interior of the cargo plane. The electrical cabinet 118 provides electrical support and routes power for the medical bio-containment module.

The first side 106 and the second side 108 include a plurality of side panels 120. The side panels are designed to withstand the forces applied through the cargo plane taking off, ascending in altitude, flying at altitude, descending in altitude, and landing. Additionally, the side panels can withstand and hold the negative pressure required within the interior of the medical bio-containment module. The side panels 120 are designed with breakaway panels that can automatically disengage from the side walls in the event of a rapid decompression situation to equalize pressure and ensure the module is safe for flight.

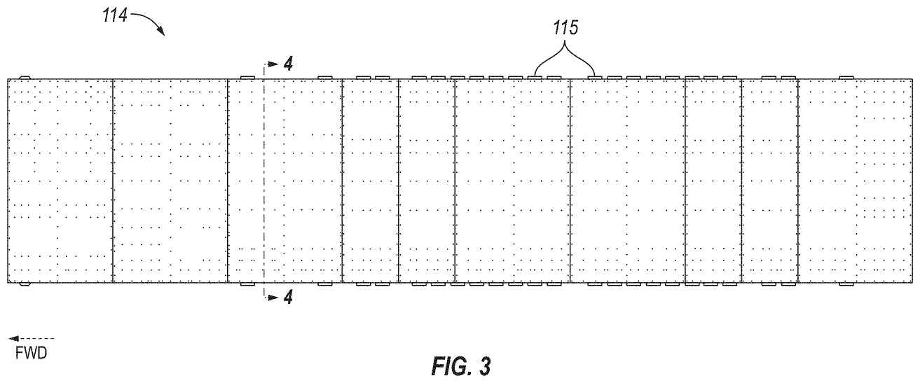

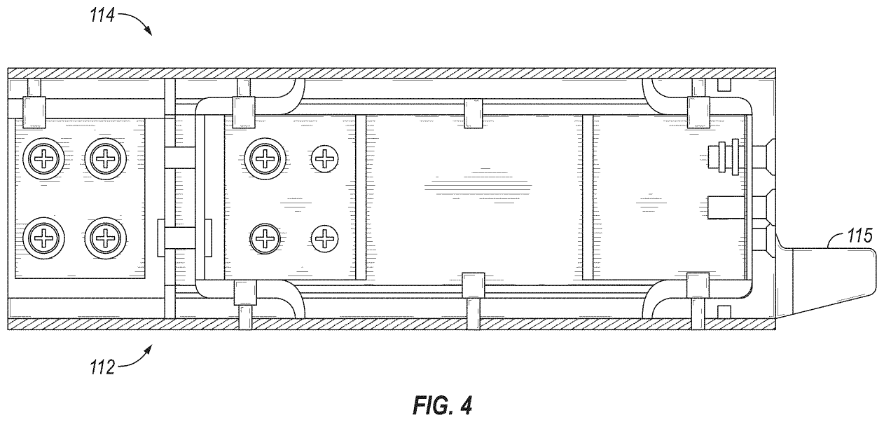

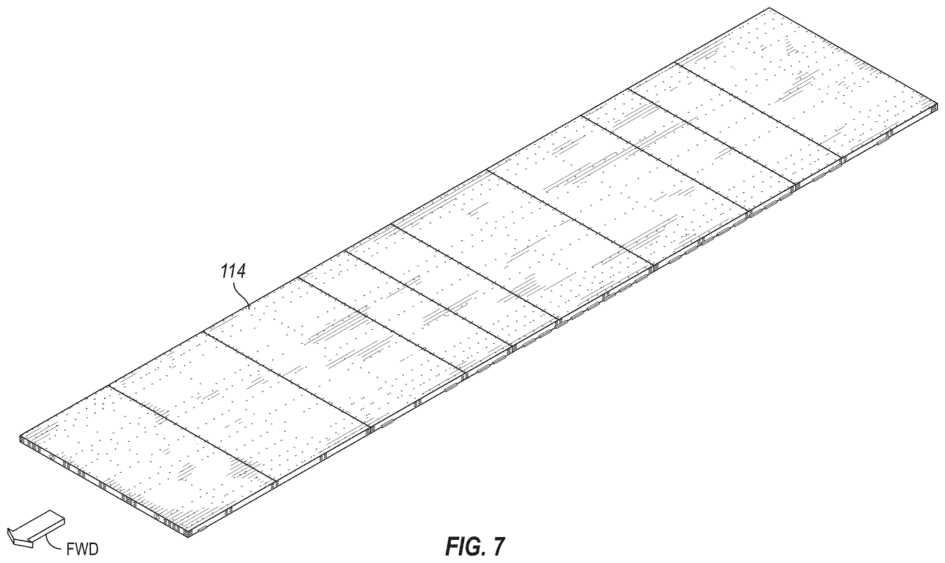

The top side 110 incudes a plurality of top panels 122. The bottom side 112 includes the pallet system 114. An embodiment of the pallet system 114 is depicted in FIGS. 3 and 7. A side view of an embodiment of the pallet system 114 is depicted in FIG. 4. The pallet system 114 is entirely integrated within the bottom side 112 of the medical bio-containment module 100, meaning that the pallet system 114 is part of, and is not separable from, the medical bio-containment module 100. The bottom of the pallet system 114 features a smooth, level surface free of bumps, large holes, or irregularities.

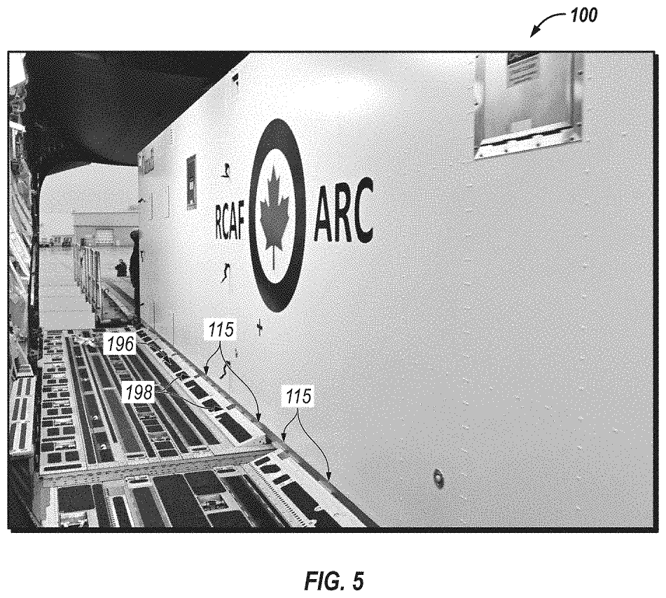

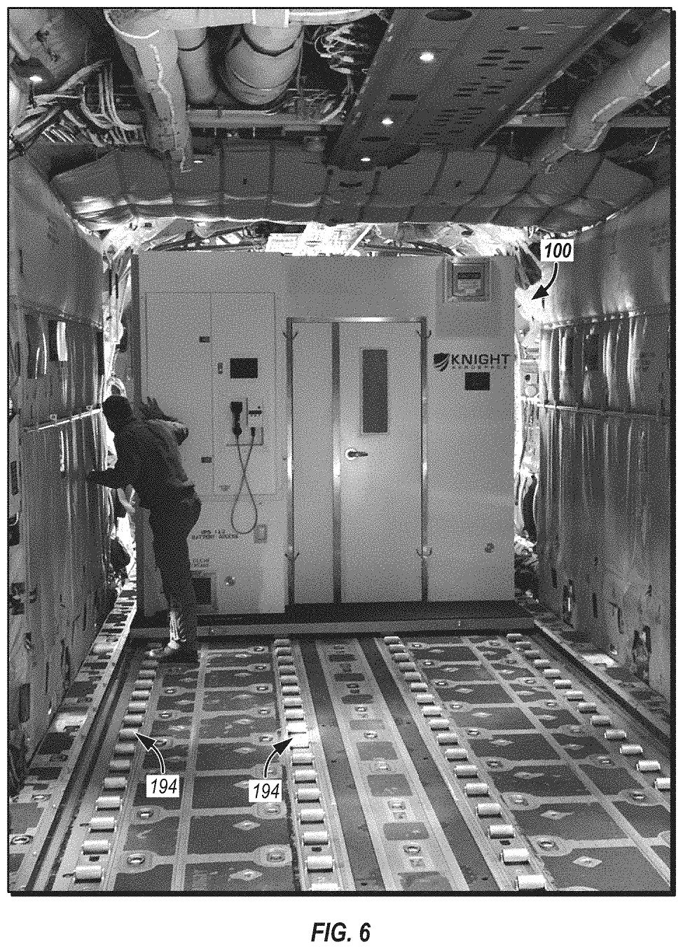

As shown in FIGS. 5 and 6, cargo planes include rollers 194 on the floor of the cargo plane, which are utilized to move items on and off the plane. The smooth surface of the bottom of the pallet system 114 allows the medical bio-containment module 100 to be rolled onto and off of the cargo plane, advantageously without having to be loaded, mounted, or affixed onto a separate pallet. When loaded on the cargo plane, the pallet system 114 of the medical bio-containment module 100 can be rolled between two side rails 196 located on the floor of the cargo plane. The medical bio-containment module 100 is designed to fit between the side rails 196 of various cargo plane configurations.

The pallet system 114 also includes lugs 115. The plurality of lugs 115 are fully integrated on the side edges of the pallet system 114, as shown in FIG. 4, which means they are not detachable from the pallet system 114. Cargo planes feature a locking system 198 incorporated into the side rails 196 or the cargo plane floor which features hook-like apparatuses that extend from the side rails 196 or the cargo plane floor. A switch in the locking system 198 engages with the lugs 115 of the pallet system 114, and this action locks the medical bio-containment module 100 in place. The plurality of lugs 115 interacts with the locking system, preventing movement or shifting of the medical bio-containment module 100 without additional chains, straps, extraneous locks, or other external equipment not fully integrated within the cargo plane or the medical bio-containment module 100.

The bottom side 112 of the medical bio-containment module 100 also includes access panels as required for access to landing gear. 4 locations are strategically placed to allow access to landing gear in the event of malfunctions in the C-130 aircraft.

All of the components encompassing the exterior of the medical bio-containment module are constructed such that the system is entirely sealed, preventing air flow into or outside of the module. The system is mechanically sealed, not welded. The exterior of the medical bio-containment module 100 is assembled with rivets, regular fasteners, and sealant. The sealant is added at every skin joint and the fasteners to seal the medical bio-containment module, which eliminates the need for welding at the intersections, joints, or fasteners. This allows the medical bio-containment module 100 is not a standard pressure vessel like a commercial aircraft. The enclosed environment includes lighting, environmental controls such as heating and air conditioning, hot water, vibration control, sound dampening, refrigeration, air quality components, and patient monitoring. As this is a completely sealed system, the patients are transported separated from the cargo hold area, and not exposed to the cargo plane cabin, which differs significantly from conventional medical evacuation systems currently utilized.

The medical bio-containment module 100 also includes a fully integrated electrical system that hooks into the cargo plane electrical system. The electrical system is designed to use MIL-STD or DO-160 approved equipment to eliminate EMI (Electrical Magnetic Interference) and RFI (Radio Frequency Interference) issues and prevent disturbances generated by an external source that affects electrical systems by electromagnet induction, electrostatic coupling, or conduction. In some embodiments, modifications may be performed to eliminate electrical interference in the different cargo aircraft models. The medical bio-containment module 100 includes receptacles with backup power to charge and support the medical equipment, not only within the ward area 270, but also within the anteroom 250 and the office area 230. Backup power is provided the maintain the negative pressure if the aircraft loses electrical power. When main power is lost, the backup power system provides power to certain systems for at least one hour. When power is restored, the power charges the batteries for the backup power system.

Other features of the medical bio-containment module 100 include the placement of acoustic materials within the structure to reduce noise from the aircraft, reducing stress and interference for the personnel and patients. All doors interior and exterior to the medical bio-containment module 100 can be removed in the event of an emergency or malfunction. Signage and placards are provided which meet FAA FAR 25 regulations. All materials, hardware, and equipment within the medical bio-containment module 100 are aircraft grade and capable of being used on ground, during take-off and landing, and during ascending and descending, and at altitude.

### (B) The Ward Area

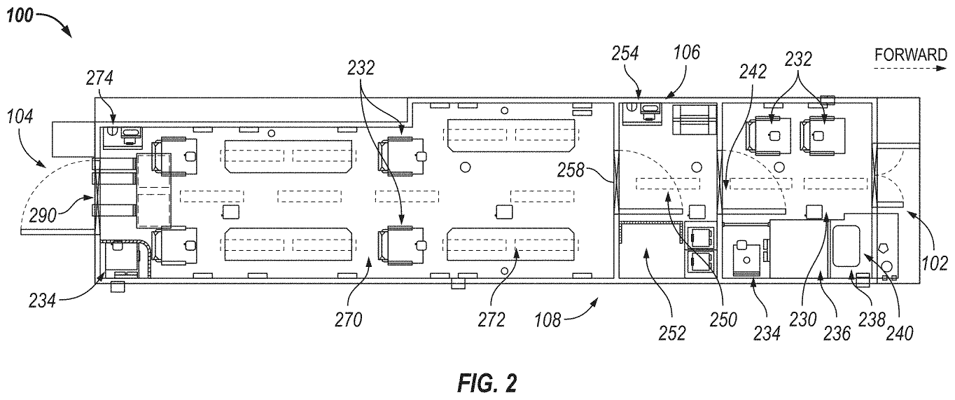

Referring to FIG. 2, the interior of the medical bio-containment module 100 is shown from an overhead view. At the back end 104, the back end door 290 allows for access to the interior of the medical bio-containment module 100. The back end door 290 is about 42″ wide to allow for patients to be loaded into the module if the patient is being carried on a NATO stretcher. Patients are loaded into and treated in the ward area 270. One or more chairs 232 are provided for personnel, located at the ends of one or more beds 272. In some embodiments, 4 chairs 232 are provided for personnel. In some embodiments, there are beds 272 spaced apart within the ward area 270. In some embodiments, 4 critical care-type beds are provided within the ward area 270. In some embodiments, there are multiple beds spaced on top of one another. In some embodiments, 3 patient litter systems holding 12 patients can be provided in the ward area 270. In other embodiments, 18 ambulatory patients can be transported in economy class airline seats. Advantageously, the medical bio-containment module can be modified to accommodate a number of different patient transportation needs. In some embodiments, the configuration of seats and beds can be changed. In some embodiments, the beds 272 can be removed and replaced with seats. The beds 272 are attached to tracks that allow the beds 272 to be pushed inward towards the aisle while still being safely secured, advantageously allowing personnel 360 degree access to the patients while preventing the beds 272 from dangerous movement in flight. The beds 272 and the chairs 232 are secured to the structure of the medical bio-containment module 100 with equipment meeting all FAA standards. The plurality of beds 272 and the plurality of chairs 232 also include a number of restraints, such as seat belts, to safely secure occupants, meeting air safety standards. Equipment to support patients is positioned on the walls of the interior and near the beds 272. Medical equipment is mounted to railings attached to the walls of the interior of the ward area 270 at each patient bed, preventing medical equipment from being mounted or placed on top of patients beds or patients themselves.

The ward area 270 also includes lavatory 234, which features a sink and a toilet for patients and personnel. The ward area 270 also includes a first sanitation station 274. The first sanitation station 274 includes hand sanitizers, surgical gloves, and a storage area. Entry to the ward area 270 can be obtained through anteroom door 258.

Equipment in the ward area 270, including all medical equipment, is rated and specially designed for use in aircraft and in flight. All materials, hardware, and equipment within the ward area 270 are aircraft grade and capable of being used on ground, during take-off and landing, and during ascending and descending, and at altitude. The chairs 232 are approved by the FAA and the TSO. The beds 272 are approved by the FAA. All materials meet current FAA requirements. An oxygen system is provided to support both personnel and patients. A liquid oxygen system is provided which supplies 4 to 6 patients with enough oxygen for up to a 15 hour mission.

The ward area 270 is under a negative air pressure system which keeps the area under a lower pressure than the areas exterior of the negative air pressure system. This negative air pressure prevents air from escaping the area, and keeps all air containments, including particulates, pathogens (including bacteria, fungi, viruses, and other airborne or droplet carried pathogens), and other containments from escaping the space.

All air exiting the medical bio-containment module 100 is filtered through a filtration system. The filtration system includes a dual fan system, multiple dust filters, and HEPA filters. The filtration system removes pathogens, particulates, droplets, and other airborne contaminants, and results in the generation of clean filtered air.

Air from outside the medical bio-containment module 100 is suctioned through fans and transported into the office area 230, then into the anteroom 250, and then into the ward area 270, which is sealed off from the rest of the medical bio-containment module and the cargo plane. The air from the ward area 270 then proceeds through the filtration system, which removes pathogens, particulates, droplets, and other contaminants from the air. The clean, filtered air is then released outside of the medical bio-containment module. The air within the medical bio-containment module 100 is recycled approximately 12 times per hour.

### (C) The Anteroom

The medical bio-containment module 100 includes the anteroom 250. The anteroom 250 transverses the medical bio-containment module connecting the office area 230 with the ward area 270. The anteroom 250 includes the anteroom door 258 which allows for entry to the ward area 270 and the office area door 242 which allows entry to the office area 230. The anteroom door 258 and the office area door 242 completely seal off the anteroom 250 when closed. The anteroom 250 is under negative air pressure, but the ward area 270 has a greater negative air pressure to prevent contamination.

The anteroom 250 includes the dumpster 252 which collects garbage and hazardous materials, including biomedical waste. The anteroom 250 also includes storage and second sanitation station 254. The second sanitation station 254 includes hand sanitizers, surgical gloves, and a storage area. The anteroom 250 also includes a bench. All materials, hardware, and equipment within the anteroom 250 are aircraft grade and capable of being used on ground, during take-off and landing, and during ascending and descending, and at altitude.

The anteroom 250 is part of the negative air pressure system, and is maintained at a pressure lower than the cargo plane. Air from the anteroom 250 is removed from the anteroom 250 and treated in the filtration system. The anteroom 250 includes a system that can be activated to inform personnel within the anteroom 250 when all air from inside the anteroom 250 has been filtered and replaced with clean air, thus indicating it is safe to remove PPE and exit the anteroom 250 into the office area 230. An access light system is installed within the anteroom 250, which indicates to personnel that the air within the anteroom has been cycled through the filtration system. The access light shows red when activated. The access light system is connected to a timer, the amount of time programmed in the timer is calculated by the amount of time it takes to purge and replace the air volume in the anteroom. Once the timer is finished, the access light turns green, indicating to personnel that it is clear to move from possible contaminated areas to clean areas, such as the office area 230.

During flights, personnel treating patients in the ward area 270 can enter the anteroom 250 through the anteroom door 258. Once the door is opened, the anteroom 250 is contaminated with air from the ward area 270, but with the office area door 242 closed the office area 230 is still sealed. Closing the anteroom door 258 allows the anteroom 250 to be isolated from the other areas. Once isolated, the negative air pressure and air filtration system will clean the air within the anteroom 250. After a specified period of time after which all of the air within the anteroom 250 is cleaned through the filtration system, a system can indicate when it is safe for personnel to remove PPE and exit the anteroom 250. This system prevents contamination from entering other areas of the module, including the office area 230.

### (D) The Office Area

The office area 230 is positioned near the front end 102 of the medical bio-containment module. The office area 230 is accessed from the anteroom 250 from the office area door 242, and from the cargo plane interior through access doors 116. The office area 230 includes a lavatory 234, which includes a toilet and a sink. The chairs 232 include a number of restraints, such as seat belts, to safely secure occupants, meeting air safety standards. The office area also includes an audio/visual system connected to the cameras in the ward area 270. The audio/visual system allows personnel to monitor patients remotely. A status panel allows personnel to see information on power failures, smoke detections, air quality, and fan operations. An electrical cabinet 240 is also within the office area 230 and allows access to electrical equipment as necessary. Also included within the office area 230 is a cooler 238 for storage of blood and other supplies or provisions requiring cool storage temperatures; a sink cabinet 236 containing a sink, a narcotics safe, and other equipment; overhead reading lights; electrical outlets; and miscellaneous other equipment. All materials, hardware, and equipment within the office area 230 are aircraft grade and capable of being used on ground, during take-off and landing, and during ascending and descending, and at altitude. The office area 230 is under a greater pressure than the anteroom 250 and the ward area 270. In some embodiments, the office area 230 is not under a negative air pressure.

1. A medical bio-containment module for use in a cargo plane, the medical bio-containment module comprising:
a pallet system comprising a flat pallet bottom, operable to allow the medical bio-containment module to be rolled on and rolled off of the cargo plane;
four walls, a floor, and a ceiling, constructed such that an airtight module interior is formed, and an outer structure of the medical bio-containment module is generated;
a negative pressure fan system, operable to maintain a negative pressure within the airtight module interior and operable to, when engaged, remove contaminated air from the medical bio-containment module and replace the contaminated air with clean filtered air;
an air filtration system connected to the negative pressure fan system, operable to remove airborne pathogens and particulates from the air;
an anteroom, comprising an office door and an anteroom door, the anteroom contained under the negative pressure from the negative pressure fan system;
a ward area, comprising the anteroom door, the ward area further comprising a patient bed, a chair, and medical equipment, the ward area contained under the negative pressure from the negative pressure fan system; and
an office area, comprising the office door and an access door.

2. The medical bio-containment module of claim 1, wherein the medical bio-containment module is operable to maintain the negative air pressure within the ward area and the anteroom of the airtight module interior through the negative pressure fan system while the cargo aircraft is in flight and while the cargo aircraft is on the ground.

3. The medical bio-containment module of claim 1, wherein the office area is not contained under the negative pressure from the negative pressure fan system.

4. The medical bio-containment module of claim 1, wherein the outer structure of the comprises aircraft grade aluminum.

5. The medical bio-containment module of claim 1, wherein the airtight module interior comprises acoustic reduction materials operable to reduce noise levels.

6. The medical bio-containment module of claim 1, wherein the pallet system further comprises a plurality of lugs, operable to allow a locking mechanism of the cargo plane to interact with the lugs and lock the medical bio-containment module in place on the cargo plane.

7. The medical bio-containment module of claim 6, wherein no chains or straps are used to lock the medical bio-containment module in place on the cargo plane.

8. The medical bio-containment module of claim 1, further comprising a decompression panel operable to blow out from the outer structure during a decompression emergency allowing air flow through the airtight module interior.

9. The medical bio-containment module of claim 1, further comprising an access panel operable to, when opened, allow access to a main landing gear in an C-130J cargo plane.

10. The medical bio-containment module of claim 1, wherein the medical bio-containment module meets or exceeds FAA FAR 25 regulations.

11. The medical bio-containment module of claim 1, wherein the chair and the patient bed are FAA approved systems.

12. The medical bio-containment module of claim 1, further comprising an electrical system operable to provide power to the medical bio-containment module, wherein the electrical system uses MIL-STD or DO-160 approved equipment.

13. The medical bio-containment module of claim 1, wherein the air filtration system further comprises dust filters and HEPA filters.

14. The medical bio-containment module of claim 1, further comprising a backup power system operable to power the medical bio-containment module in the event of a cargo plane power source is lost.

15. The medical bio-containment module of claim 1, wherein the medical equipment comprises a liquid oxygen system operable to provide patients with oxygen for a 15 hour mission.

16. A method of using the medical bio-containment module of claim 1, the method comprising the steps of:
rolling the medical bio-containment module onto the cargo aircraft;
locking the medical bio-containment module onto the cargo plane by engaging the locking system on the cargo plane such that the locking system interacts with a plurality of lugs, the pallet system comprising the plurality of lugs;
connecting an electrical system from the medical bio-containment module to a cargo aircraft electrical system;
loading patients into the ward area;
shutting the access door, the office door, and the anteroom door; and
engaging the negative pressure fan system.

17. The method of claim 16, wherein the step of locking the medical bio-containment module onto the cargo plane is performed in the absence of the use of chains or straps.
